(19) United States
(12) Patent Application Publication
(10) Pub. No.: US 2010/0076516 A1
(43) Pub. Date: Mar. 25, 2010

(54) REMAINING TIME INDICATION FOR A RECHARGEABLE IMPLANTABLE MEDICAL DEVICE

(75) Inventors: Alexander Padiy, Geldrop (NL); Hubert Cecile Francois Martens, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/442,740

(22) PCT Filed: Sep. 20, 2007
(86) PCT No.: PCT/IB07/53832

§ 371 Date: Mar. 25, 2009

Related U.S. Application Data
(60) Provisional application No. 60/826,915, filed on Sep. 26, 2006.

(51) Int. Cl. A61N 1/08

(52) U.S. Cl. 607/29; 607/2

(57) ABSTRACT

An implantable medical device for delivering a therapeutic output to a patient, comprising: a rechargeable electrical power source having a useful life; a therapeutic delivery device operatively coupled to the power source and adapted to deliver the therapeutic output to the patient; a power source recharge timing indicator operatively coupled to the power source, wherein the timing indicator includes means for determining and communicating when the remaining usage time before full drainage of the power source drops below a first predetermined level based on measurement of one or more physical characteristics of the power source and of the medical device; and safe mode means operatively coupled to the timing indicator, power source and therapeutic delivery device, wherein the safe mode means, upon activation, is capable of causing one or more actions to reduce the power consumption of the medical device; wherein the safe mode means is activated by receiving communications from the timing indicator that the remaining usage time before full drainage of the power source has dropped below one or more second predetermined levels, thereby preventing excessive power drainage from the power source which would result in damage to the power source and/or medical device and/or injury to the patient. A method for preventing excessive power drainage and indicating the remaining discharge time of the power source of an implantable medical device for delivering a therapeutic output to a patient, which would result in damage to the power source and/or medical device and/or injury to the patient is also disclosed.

The disclosure is directed to an implantable medical device for delivering a therapeutic output to a patient having a rechargeable electrical power source having a useful life; a therapeutic delivery device operatively coupled to the power source and adapted to deliver the therapeutic output to the patient; a power source recharge timing indicator for monitoring the remaining usage time before full drainage of the power source; and safe mode means to reduce the power consumption of the medical device, thereby preventing excessive power drainage from the power source which would result in damage to the power source and/or medical device and/or injury to the patient.

Implantable medical devices such as implantable cardiac pacemakers, implantable cardiac defibrillators, implantable drug pumps or infusion devices, implantable neurostimulation devices, cochlear implants, or implantable neuroprostheses are becoming increasingly more often used in clinical practice. In many cases, the devices need to remain inside a patient's body for prolonged periods (e.g. years) while remaining fully functioning. As such, a battery must be provided with the implant that is able to supply the required energy for the operation of the device while being inside the body. Since often the devices should be able to operate for very long times, the volume of the battery is rather large (10 s of cm³) in order to store the required energy. As a result the implantable devices cannot be easily reduced in size. This is disadvantageous because smaller implants are likely to be more easily inserted in a patient's body, e.g. using minimally invasive surgery techniques. Furthermore, there are less restrictions on the location of the implant when its size is smaller.

A recent development is the use of rechargeable batteries (e.g. Li-ion) in implantable medical devices. Since the battery may be recharged regularly, e.g. once a week, the total energy that needs to be stored is much less and, concomitantly, the battery volume can be reduced. Implants with rechargeable batteries therefore can be made significantly smaller in volume than those with non-rechargeable batteries. When the battery is drained, the device will cease to function. Although this is not necessarily a life-threatening situation in case of implants that do not provide a vital function, in the case of e.g. cardiac devices a potentially dangerous situation may occur for the patient. In case of spinal cord stimulation (SCS) devices, the patient will notice the pain returns because the stimulation is ceased, and he/she will be immediately urged to recharge the device. However, in case of e.g. cardiac defibrillator the patient may not notice that the device has ceased operation until it is too late.

In addition, when the battery is not recharged on time, or not replaced on time, it is possible that the battery is fully drained during in vivo operation of the device, so the device will cease operation. This may lead to harmful situations for the patient. For instance, in case of a drug pump, the drug flow may not be controlled anymore, which could lead to excessive drug supply with serious consequences, e.g. poisoning. In case of electrical stimulation devices (e.g. deep brain stimulation (DBS) or cardiac devices), active protection of e.g. RF interference on the conducting leads could become disabled, which could result in injuries to the patient in case RF fields are picked up by the leads. Excessive drainage of the rechargeable battery may also prevent the device from initiating the recharging procedure because the device cannot power up the necessary circuits, which will necessitate replacement of the implantable device or the rechargeable battery. A different problem is that excessive drainage of a rechargeable battery may also damage the battery itself, which is also unwanted, as it again necessitates the replacement of the battery or the device. It is noted that replacement of the implantable device or its battery is unwanted in general since it requires additional surgery.

Various systems and methods for monitoring the status of power sources in medical devices have been disclosed heretofore. For example, U.S. Pat. No. 5,127,402 granted Jul. 7, 1992, European patent application 1,610,437A1 published Dec. 28, 2005 and U.S. Patent Application 20030114899 published Jun. 19, 2003 disclose systems for transferring implanted medical devices into lower power consumption modes. Also, U.S. Pat. No. 6,901,293 granted May 31, 2005 discloses a system for monitoring power source longevity in an implantable medical device.

However, there are still many shortcomings in such systems and methods, particularly with regard to implanted medical devices having rechargeable power sources and with regard to preventing serious injury to patients if the power source is excessively drained and damaged.

Therefore, there is a need for a rechargeable implantable medical device and method that can timely inform the need for recharging of the power source (e.g., battery) in an implanted medical device in order to guarantee the proper functioning of the device, as well as to be able to power down the medical device to one or more “safe mode” levels of lower power consumption operation of the medical device to prevent irreversible damage to the power source and/or implantable device and/or injury to the patient.

These and other needs are satisfied with the rechargeable implantable medical device of the present disclosure.

According to the present disclosure, an implantable medical device for delivering a therapeutic output to a patient having a rechargeable electrical power source having a useful life; a therapeutic delivery device operatively coupled to the power source and adapted to deliver the therapeutic output to the patient; a power source recharge timing indicator for monitoring the remaining usage time before full drainage of the power source; and safe mode means to reduce the power consumption of the medical device, thereby preventing excessive power drainage from the power source which would result in damage to the power source and/or implantable device and/or injury to the patient is disclosed.

Specifically, it is an object of the invention to provide an implantable medical device for delivering a therapeutic output to a patient, comprising:
a rechargeable electrical power source having a useful life;
a therapeutic delivery device operatively coupled to the power source and adapted to deliver the therapeutic output to the patient;
a power source recharge timing indicator operatively coupled to the power source, wherein the timing indicator includes means for determining and communicating when the remaining usage time before full drainage of the power source drops below a first predetermined level based on measurement of one or more physical characteristics of the power source and of the medical device; and
safe mode means operatively coupled to the timing indicator, power source and therapeutic delivery device, wherein the safe mode means, upon activation, is capable of causing one or more actions to reduce the power consumption of the medical device; wherein the safe mode means is activated by receiving communications from the timing indicator that the remaining usage time before full drainage of the power source has dropped below one or more second predetermined levels, thereby preventing excessive power drainage from the power source which would result in damage to the power source and/or medical device and/or injury to the patient.

Another object is to provide a medical device wherein the power source is a rechargeable battery, preferably a rechargeable lithium-ion battery.

Another object is to provide a medical device wherein the measured physical characteristics of the battery are voltage, impedance or current.

Another object is to provide a medical device wherein the implantable medical device is selected from the group consisting of cardiac pacemakers, cardiac defibrillators, drug infusion devices, neurostimulation devices, cochlear implants, neuroprosthetic devices and combinations thereof.

Another object is to provide a medical device wherein each of the safe mode means and the timing indicator are independently capable of communicating with each other, and one or more of the patient, an external programming device and an external operator.

Another object is to provide a medical device wherein, upon activation, the safe mode means is capable of causing one or more of the following actions to occur affecting the medical device:
discontinuing delivery of a drug to the patient;
switching the electrical leads of the medical device to a high impedance state to prevent undesirable interference from external RF signals;
switching to a lower power consumption mode by switching from closed-loop therapy-delivery mode to open-loop therapy-delivery mode and switching down sensing/feedback circuitry;
switching to a lower power consumption mode of stimulation and/or sensing;
fully stopping the stimulation and/or sensing;
switching to intermittent mode of stimulation and/or sensing;
storing parameters of the medical device and remaining usage time in a non-volatile memory;
powering down all of the circuitry in the medical device except for the circuitry for recharging the power source; and
fully powering down the medical device in order to prevent destruction of the power source.

Another object is to provide a medical device wherein the timing indicator is capable of communicating a signal that can be sensed by at least one of the patient, the safe mode means, an external programming device, and an external operator when the remaining usage time falls below the first predetermined level.

Another object is to provide a medical device wherein the first and/or the one or more second predetermined levels can be programmed by an external operator or external programming device.

Another object is to provide a medical device wherein the signal communicated by the timing indicator to the patient can be sensed by the patient.

Another object is to provide a medical device wherein the signal is one or more of a sound, a vibration or a flashing light.

Another object is to provide a medical device wherein the first predetermined level is one of one day, two days or one week.

Another object is to provide a medical device further comprising restart means, which upon activation, is capable of restarting the device from a full power down mode to permit recharging of the power source.

Another object is to provide a medical device wherein the restart means is capable of being activated upon communication of an activating signal from an external operator.

Another object is to provide a medical device wherein the activating signal is magnetic, light or electromagnetic in origin.

Another object is to provide a method for preventing excessive power drainage and indicating the remaining discharge time of the power source of an implantable medical device for delivering a therapeutic output to a patient, which would result in damage to the power source and/or medical device and/or injury to the patient, the method comprising:

implanting the medical device in the patient, the medical device comprising:
a rechargeable electrical power source having a useful life;
a therapeutic delivery device operatively coupled to the power source and adapted to deliver the therapeutic output to the patient;
a power source recharge timing indicator operatively coupled to the power source, wherein the timing indicator includes means for determining and communicating when the remaining usage time before full drainage of the power source drops below a first predetermined level based on measurement of one or more physical characteristics of the power source and of the medical device; and
safe mode means operatively coupled to the timing indicator, power source and therapeutic delivery device, wherein the safe mode means, upon activation, is capable of causing one or more actions to reduce the power consumption of the medical device; wherein the safe mode means is activated by receiving communications from the timing indicator that the remaining usage time before full drainage of the power source has dropped below one or more second predetermined levels, thereby preventing excessive power drainage from the power source;
determining and communicating when the remaining usage time before full drainage of the power source drops below a first predetermined level based on measurement of one or more physical characteristics of the power source and of the medical device; and
causing one or more actions to reduce the power consumption of the medical device; wherein the safe mode means is activated by receiving communications from the timing indicator that the remaining usage time before full drainage of the power source has dropped below one or more second predetermined levels, thereby preventing excessive power drainage from the power source which would result in damage to the power source and/or medical device and/or injury to the patient.

Another object is to provide a method wherein the power source is a rechargeable battery, preferably a rechargeable lithium-ion battery.

Another object is to provide a method wherein the measured physical characteristics of the battery are voltage, impedance or current.

Another object is to provide a method wherein the implantable medical device is selected from the group consisting of cardiac pacemakers, cardiac defibrillators, drug infusion devices, neurostimulation devices, cochlear implants, neuroprosthetic devices and combinations thereof.

Another object is to provide a method wherein each of the safe mode means and the timing indicator are independently capable of communicating with each other, and one or more of the patient, an external programming device and an external operator.

Another object is to provide a method wherein, upon activation, the safe mode means is capable of causing one or more of the following actions to occur affecting the medical device:
discontinuing delivery of a drug to the patient;
switching the electrical leads of the medical device to a high impedance state to prevent undesirable interference from external RF signals;
switching to a lower power consumption mode by switching from closed-loop therapy-delivery mode to open-loop therapy-delivery mode and switching down sensing/feedback circuitry;
switching to a lower power consumption mode of stimulation and/or sensing;
fully stopping the stimulation and/or sensing;
switching to intermittent mode of stimulation and/or sensing;
storing parameters of the medical device and remaining usage time in a non-volatile memory;
powering down all of the circuitry in the medical device except for the circuitry for recharging the power source; and
fully powering down the medical device in order to prevent destruction of the power source.

Another object is to provide a method wherein the timing indicator is capable of communicating a signal that can be sensed by at least one of the patient, the safe mode means, an external programming device, and an external operator when the remaining usage time falls below the first predetermined level.

Another object is to provide a method wherein the first and/or the one or more second predetermined levels can be programmed by an external operator or external programming device.

Another object is to provide a method wherein the signal communicated by the timing indicator to the patient can be sensed by the patient.

Another object is to provide a method wherein the signal is one or more of a sound, a vibration or a flashing light.

Another object is to provide a method wherein the first predetermined level is one of one day, two days or one week.

Another object is to provide a method further comprising restart means, which upon activation, is capable of restarting the device from a full power down mode to permit recharging of the power source.

Another object is to provide a method wherein the restart means is capable of being activated upon communication of an activating signal from an external operator.

Another object is to provide a method wherein the activating signal is magnetic, light or electromagnetic in origin.

These and other aspects of the invention are explained in more detail with reference to the following embodiments and with reference to the figures.

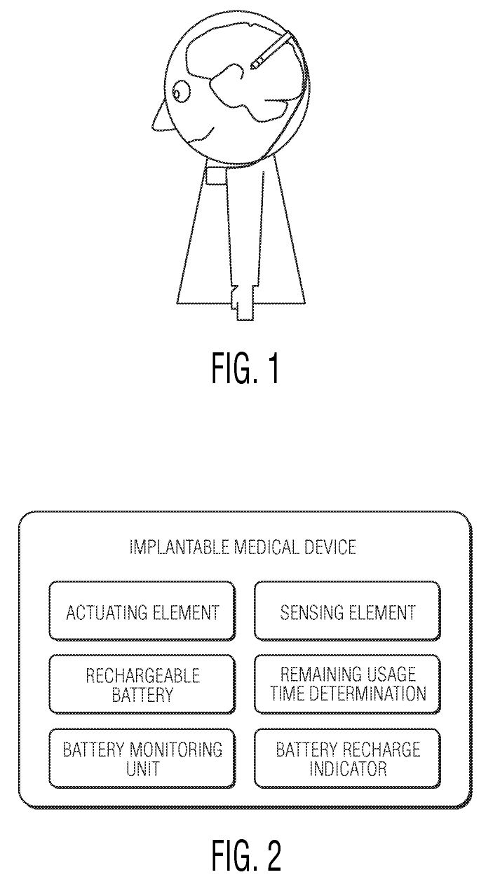

FIG. 1 depicts an example of an implantable medical device which is a deep brain stimulation unit.

FIG. 2 is a flow chart illustrating the functions of an implantable medical device according to an embodiment of the invention.

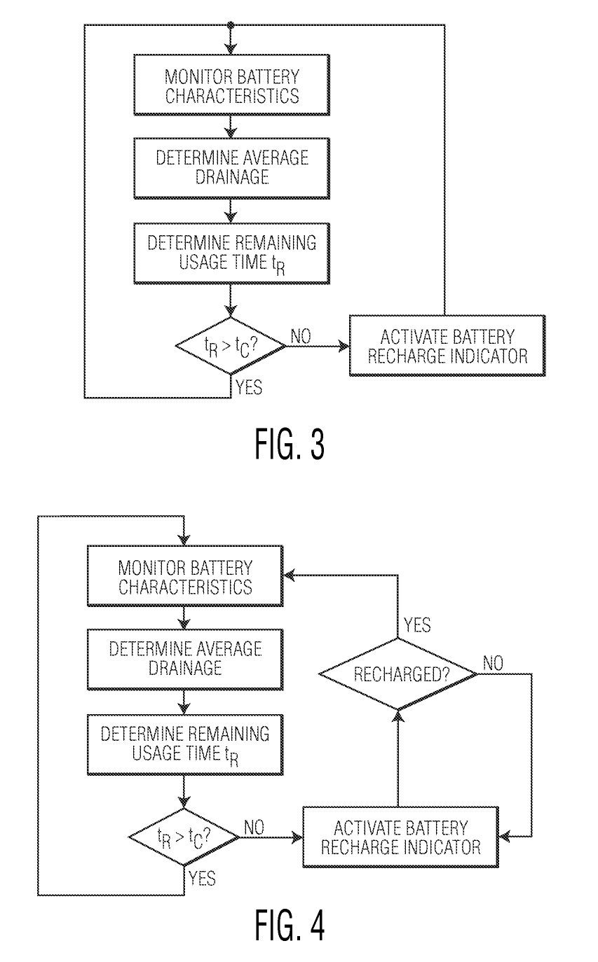

FIG. 3 is a flow chart illustrating the method for a battery recharge indicator in an implantable medical device according to an embodiment of the invention.

FIG. 4 is a flow chart illustrating the method for a battery recharge indicator in an implantable medical device according to an embodiment of the invention.

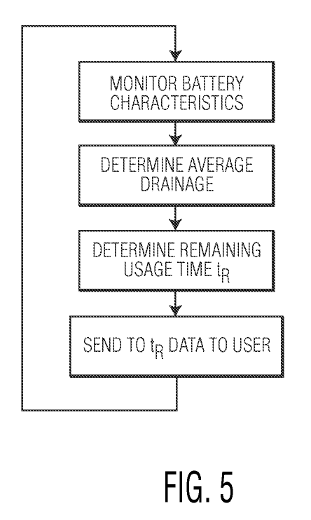

FIG. 5 is a flow chart illustrating the method to communicate remaining time to a user, not using a recharge indicator in an implantable medical device according to an embodiment of the invention.

Implantable medical devices having a power source, for treating a variety of conditions in a patient are well known. One type of medical device is an implantable therapeutic substance infusion device or drug pump. An implantable therapeutic substance infusion device is implanted by a clinician into a patient at a location appropriate for the therapy. Typically, a therapeutic substance infusion catheter is connected to the device outlet and implanted to infuse the therapeutic substance such as a drug or infusate at a programmed infusion rate and predetermined location to treat a condition such as pain, spasticity, cancer, and other medical conditions.

Other examples of implantable devices are heart defibrillators, pacemakers and those which electrically stimulate neurological tissue to treat or relieve the symptoms of a wide variety of physiological or psychological maladies or pain. Such devices are typically part of systems that are entirely implantable within the patient or are partially implantable and partially external to the patient. Systems that are entirely implantable in the patient typically include an implantable pulse generator and an extension and lead or leads. In such a system, the implantable pulse generator, extension and lead are entirely implanted in the bodies of the patients. Because the implantable pulse generator is implanted, the power sources needed to power the implantable pulse generator are also implanted. Typically, the power source for an implantable pulse generator is a battery.

Each of these implantable devices delivers a therapeutic output to the patient. In the case of an implantable therapeutic substance infusion device, the therapeutic output can be a therapeutic substance which is infused into the patient. In the case of a neurological tissue stimulator, the therapeutic output is an electrical signal intended to produce a therapeutic result in the patient. Other types of implantable therapeutic delivery devices also exist including cardiac pacemakers and defibrillators.

Electrically powered implanted therapeutic delivery devices can require replacement once implanted due to factors such as battery consumption, corrosive damage and mechanical wear. Since replacement of the implanted therapeutic delivery device requires an invasive procedure of explanting the existing device and implanting a new device, it is desirable to only replace the therapeutic delivery device when replacement is required. Replacement of previously implanted therapeutic delivery devices was typically scheduled based upon a worst-case statically forecasted elective replacement period. The worst-case scenario typically resulted in the implanted therapeutic delivery device being replaced several months or even years before the implanted therapeutic delivery device actually required replacement.

Battery monitors which monitor the voltage of the battery in order to determine, or to predict, the remaining longevity of the battery have an inherent shortcoming. The voltage of a battery will commonly very slowly decline over time with only a slight variation in the voltage until the voltage the battery nears the end of its useful life. As the battery nears the end of its useful life, the battery voltage will begin to decline at a greater rate, often dramatically. Such a battery is advantageous as a source of power for an implantable therapeutic delivery device because the battery delivers such an assured relatively constant voltage over most of the useful life of the device. However, such a battery creates a problem for a battery longevity monitor using the voltage of the battery in an attempt to determine the longevity of the battery. Since the battery voltage remains relatively constant over most of the life of the battery, it is difficult to predict whether the battery is in the early part of the relatively flat voltage curve or nearing the end of the relatively flat voltage curve. The difference, of course, is a marked difference in the predicted longevity of the battery.

The ability to accurately predict the remaining longevity of the power source of an implantable therapeutic delivery device enables the patient to receive maximum life from the device and minimize the frequency, and possibly the number, of explanation and reimplantation of the device simply for the replacement of the power source. Further, since some safety margin is usually built in and because the patient usually schedules any such explanation and reimplantation, often around a busy schedule, additional time off of the actual remaining longevity of the power source may be lost. Moreover, the use of rechargeable power sources (for example, batteries) offers the advantage of being able to substantially reduce the need for surgery to the patient simply to replace the discharged battery when it is not rechargeable.

However, there then remains the challenge of indicating to the patient or external operator (for example, nurse, clinician, operator, or external programming device) that the power source needs recharging or that the power consumption should be reduced in stages to one or more safe mode levels to prevent irreparable damage to the power source and/or medical device and/or injury to the patient.

According to the systems and methodology of the invention, when battery drainage is detected, safe mode means of the medical device causes the device to be parked in a so-called “safe mode”, which may include both preventing the power source (for example, battery) from further discharging by disconnecting the power-hungry circuitry and ensuring patient safety by putting the device into a state which would allow abrupt disconnection of the battery power without compromising patient safety. Multiple grades of the “safety mode” may also be used for a more gradual transition from the operational mode to the off-mode. For example, the device may first enter a low-power mode with reduced functionality, then switch to a mode insensitive to an abrupt disconnection of power, and only then switch the device off except for the circuitry responsible for initiating the recharge session (in case of a rechargeable system). Switching between the different grades of the “safety mode” can be done either based on a physical parameter characterizing the battery state (voltage, internal resistance, optical properties, etc.) or by means of a time-out mechanism.

For example the following actions can be taken when switching to one of the “safe mode” levels:
Closing the drug reservoir;
Switching to the leads to a high impedance state (e.g. by means of bi-stable switches like MEMS);
Switching to a lower power consumption mode by switching from closed-loop therapy-delivery mode to open-loop therapy-delivery mode and switching down sensing/feedback circuitry;
Switching to lower energy consumption regime of stimulation and/or sensing;
Fully stopping the stimulation and/or sensing;
Storing device parameters and status information in a non-volatile memory;
Switching to intermittent mode of stimulation and/or sensing;
Powering down all the circuitry except for the circuitry responsible for initiating the recharge session (in case of rechargeable system); and
Fully powering the device down in order to prevent destruction of the battery.

In another aspect of the invention a recharge timing indicator or power source recharge timing indicator is incorporated into the implantable medical device that indicates the remaining usage time left before full drainage of the rechargeable power source, based on the measurement of one or more physical characteristics of the power source and of the implanted medical device. The recharge timing indicator can include means for determining and communicating this timing indicator information to a user, e.g. a patient, a nurse, a clinician, or a close relative of the patient. Thus, when the remaining time until full discharge falls below a predetermined or preprogrammed level in the medical device, the timing indicator communicates or transmits a signal to the patient (for example, by audible sound, vibration or light) that the power source needs recharging. The predetermined time level can be, for example, 1 day, 2 days, 1 week, or some other time interval). Alternatively, the communication can be made externally, for example, to a nurse, operator, clinician, external programming device. Note, that in the case of primary (i.e. non-rechargeable) batteries, the problem is solved by measuring and indicating the remaining battery lifetime before replacement. Several embodiments of the invention follow below and in the FIGS. 1-5:
In an embodiment the rechargeable power source is a rechargeable battery.
In an embodiment the rechargeable battery is a Lithium-ion battery.
In an embodiment the remaining usage time is estimated from the ratio of the state of charge of the battery and (a running average of) charge requirement by the implantable medical device.
In an embodiment the state of charge of the battery is derived from measurable physical battery parameters such as battery voltage, battery impedance, and battery voltage relaxation time (see Pop, V.; Bergveld, H. J.; Notten, P. H. L., “State-of-Charge Indication in Portable Applications,” Industrial electronics, 2005. ISIE 2005. Proceedings of the IEEE International Symposium on, vol. 3, no. pp. 1007-1012, Jun. 20-23, 2005).
In an embodiment a battery recharge indicator module is activated when the remaining usage time t_{R} drops below a critical value t_{C}.
In an embodiment t_{C} may be one, two, or more days, or a week; in another embodiment the value of t_{C} may be programmed (e.g. by a clinician) into the implantable medical device.
In an embodiment the recharge indicator communicates with an external device, e.g. a programming device.
In an embodiment the recharge indicator induces a signal that can be sensed by a user, e.g. a sound.

In another aspect of the invention, and a refinement to the concept of “safe mode” levels, the medical device can include restart means for switching the “safe mode” level or “waking up” the device after full shutdown using external signaling. The concept of external switching can be quite useful to bring the device back to life when the device goes into the “full power down” mode due to excessive battery drainage (i.e. how can it “wake up” from the “full power down” mode for starting a recharge session). To circumvent the problem, it is contemplated within the invention framework to trigger the device to “wake up” by an external device available to clinical specialists or service personnel. In particular, the device can be externally triggered to shift from the “full power down” state to the “only recharge circuitry is on” state either by means using signals from a strong magnet placed near the device, by means of shining light on the device through the skin or by means of radiating electromagnetic waves onto the device.

In case of using a magnet, mechanical (MEMS) structures sensitive to magnetic fields can be employed for performing the actual state switching.

In case of light activation, a photodiode can be used for generating the current needed for actuating a switching element that puts the device from one state to the other. Also, the light coming from the external light source can be used for supplying the power needed by the recharge circuitry at the beginning of the recharge session (when the voltage supplied by the implanted battery may be insufficient). In case of electromagnetic wave activation, the device can be equipped with a resonant LC circuit that generates sufficient current/voltage when exposed to external electromagnetic wave source of specific frequency. As in the case of activation by light, the scheme can be used for generating power during initial phases of the recharge session as well.

While the present invention has been described with respect to specific embodiments thereof, it will be recognized by those of ordinary skill in the art that many modifications, enhancements, and/or changes can be achieved without departing from the spirit and scope of the invention. Therefore, it is manifestly intended that the invention be limited only by the scope of the claims and equivalents thereof.

1. An implantable medical device for delivering a therapeutic output to a patient, comprising:
a rechargeable electrical power source having a useful life;
a therapeutic delivery device operatively coupled to the power source and adapted to deliver the therapeutic output to the patient;
a power source recharge timing indicator operatively coupled to the power source, wherein the timing indicator includes means for determining and communicating when the remaining usage time before full drainage of the power source drops below a first predetermined level based on measurement of one or more physical characteristics of the power source and of the medical device; and safe mode means operatively coupled to the timing indicator, power source and therapeutic delivery device, wherein the safe mode means, upon activation, is capable of causing one or more actions to reduce the power consumption of the medical device; wherein the safe mode means is activated by receiving communications from the timing indicator that the remaining usage time before full drainage of the power source has dropped below one or more second predetermined levels, thereby preventing excessive power drainage from the power source.

2. The medical device of claim 1 wherein the power source is a rechargeable battery.

3. The medical device of claim 2 wherein the power source is a rechargeable lithium-ion battery.

4. The medical device of claim 2 wherein the measured physical characteristics of the battery are voltage, impedance or current.

5. The medical device of claim 1 wherein the implantable medical device is selected from the group consisting of cardiac pacemakers, cardiac defibrillators, drug infusion devices, neurostimulation devices, cochlear implants, neuroprosthetic devices and combinations thereof.

6. The medical device of claim 1 wherein each of the safe mode means and the timing indicator are independently capable of communicating with each other, and one or more of the patient, an external programming device and an external operator.

7. The medical device of claim 1 wherein, upon activation, the safe mode means is capable of causing one or more of the following actions to occur affecting the medical device:
discontinuing delivery of a drug to the patient;
switching the electrical leads of the medical device to a high impedance state to prevent undesirable interference from external RF signals;
switching to a lower power consumption mode by switching from closed-loop therapy-delivery mode to open-loop therapy-delivery mode and switching down sensing/feedback circuitry;
switching to a lower power consumption mode of stimulation and/or sensing;
fully stopping the stimulation and/or sensing;
switching to intermittent mode of stimulation and/or sensing;
storing parameters of the medical device and remaining usage time in a non-volatile memory;
powering down all of the circuitry in the medical device except for the circuitry for recharging the power source; and
fully powering down the medical device in order to prevent destruction of the power source.

8. The medical device of claim 1 wherein the timing indicator is capable of communicating a signal that can be sensed by at least one of the patient, the safe mode means, an external programming device, and an external operator when the remaining usage time falls below the first predetermined level.

9. The medical device of claim 1 wherein the first and/or the one or more second predetermined levels can be programmed by an external operator or external programming device.

10. The medical device of claim 8 wherein the signal communicated by the timing indicator to the patient can be sensed by the patient.

11. The medical device of claim 10 wherein the signal is one or more of a sound, a vibration or a flashing light.

12. The medical device of claim 9 wherein the first predetermined level is one of one day, two days or one week.

13. The medical device of claim 1 further comprising restart means, which upon activation, is capable of restarting the device from a full power down mode to permit recharging of the power source.

14. The medical device of claim 13 wherein the restart means is capable of being activated upon communication of an activating signal from an external operator.

15. The medical device of claim 14 wherein the activating signal is magnetic, light or electromagnetic in origin.

16. A method for preventing excessive power drainage and indicating the remaining discharge time of the power source of an implantable medical device for delivering a therapeutic output to a patient, which would result in damage to the power source and/or medical device and/or injury to the patient, the method comprising:
implanting the medical device in the patient, the medical device comprising:
a rechargeable electrical power source having a useful life;
a therapeutic delivery device operatively coupled to the power source and adapted to deliver the therapeutic output to the patient;
a power source recharge timing indicator operatively coupled to the power source, wherein the timing indicator includes means for determining and communicating when the remaining usage time before full drainage of the power source drops below a first predetermined level based on measurement of one or more physical characteristics of the power source and of the medical device; and safe mode means operatively coupled to the timing indicator, power source and therapeutic delivery device, wherein the safe mode means, upon activation, is capable of causing one or more actions to reduce the power consumption of the medical device; wherein the safe mode means is activated by receiving communications from the timing indicator that the remaining usage time before full drainage of the power source has dropped below one or more second predetermined levels, thereby preventing excessive power drainage from the power source;
determining and communicating when the remaining usage time before full drainage of the power source drops below a first predetermined level based on measurement of one or more physical characteristics of the power source and of the medical device; and
causing one or more actions to reduce the power consumption of the medical device; wherein the safe mode means is activated by receiving communications from the timing indicator that the remaining usage time before full drainage of the power source has dropped below one or more second predetermined levels, thereby preventing excessive power drainage from the power source.

17. The method of claim 16 wherein the power source is a rechargeable battery.

18. The method of claim 17 wherein the power source is a rechargeable lithium-ion battery.

19. The method of claim 17 wherein the measured physical characteristics of the battery are voltage, impedance or current.

20. The method of claim 16 wherein the implantable medical device is selected from the group consisting of cardiac pacemakers, cardiac defibrillators, drug infusion devices, neurostimulation devices, cochlear implants, neuroprosthetic devices and combinations thereof.

21. The method of claim 16 wherein each of the safe mode means and the timing indicator are independently capable of communicating with each other, and one or more of the patient, an external programming device and an external operator.

22. The method of claim 16 wherein, upon activation, the safe mode means is capable of causing one or more of the following actions to occur affecting the medical device:
discontinuing delivery of a drug to the patient;
switching the electrical leads of the medical device to a high impedance state to prevent undesirable interference from external RF signals;
switching to a lower power consumption mode by switching from closed-loop therapy-delivery mode to open-loop therapy-delivery mode and switching down sensing/feedback circuitry;
switching to a lower power consumption mode of stimulation and/or sensing;
fully stopping the stimulation and/or sensing;
switching to intermittent mode of stimulation and/or sensing;
storing parameters of the medical device and remaining usage time in a non-volatile memory;
powering down all of the circuitry in the medical device except for the circuitry for recharging the power source; and
fully powering down the medical device in order to prevent destruction of the power source.

23. The method of claim 16 wherein the timing indicator is capable of communicating a signal that can be sensed by at least one of the patient, the safe mode means, an external programming device, and an external operator when the remaining usage time falls below the first predetermined level.

24. The method of claim 16 wherein the first and/or the one or more second predetermined levels can be programmed by an external operator or external programming device.

25. The method of claim 23 wherein the signal communicated by the timing indicator to the patient can be sensed by the patient.

26. The method of claim 25 wherein the signal is one or more of a sound, a vibration or a flashing light.

27. The method of claim 24 wherein the first predetermined level is one of one day, two days or one week.

28. The method of claim 16 further comprising restart means, which upon activation, is capable of restarting the device from a full power down mode to permit recharging of the power source.

29. The method of claim 28 wherein the restart means is capable of being activated upon communication of an activating signal from an external operator.

30. The method of claim 29 wherein the activating signal is magnetic, light or electromagnetic in origin.
